(19) United States
(12) Patent Application Publication
(10) Pub. No.: US 2008/0267939 A1
(43) Pub. Date: Oct. 30, 2008

(54) SYNERGISTIC ANTI-HYPERTENSIVE PHYTO-NUTRACEUTICAL COMPOSITION

(76) Inventor: JOSE ANGEL OLALDE RANGEL, Clearwater, FL (US)

Correspondence Address: JOSE OLALDE RANGEL, 519 CLEVELAND STREET, SUITE 101, CLEARWATER, FL, 33755, US

(21) Appl. No.: 11/536,776

(22) Filed: Sep. 29, 2006

(51) Int. Cl. A61K 36/258; A61K 36/73; A61K 36/16; A61K 36/81; A61K 125/00; A61K 33/06; A61P 9/12; A61K 31/122

(52) U.S. Cl. 424/94.1; 424/728; 424/765; 424/752; 424/760; 424/773; 424/682; 514/689

(57) ABSTRACT

A Phyto-nutraceutical composition for the prevention and treatment of hypertensive disorders is provided. A specific combination of extracts of plants and nutraceuticals is provided, based on categorizing plants and nutraceuticals into one of three groups, Energy, Bio-Intelligence, and Organization. Such combinations have synergistic effects, with minimal side effect.

### PRIOR RELATED APPLICATIONS

Not applicable.

### FEDERALLY SPONSORED RESEARCH STATEMENT

Not applicable.

### REFERENCE TO MICROFICHE APPENDIX

Not applicable.

### FIELD OF THE INVENTION

The invention relates to a phytoceutical formulation used to treat hypertensive disorders. The formulation is a particular combination of plants and nutraceuticals and has a synergistic effect in combination.

### BACKGROUND OF THE INVENTION

The academic study of medicinal plants for the treatment of diverse diseases has been nearly as pervasive as the study of Western medicines. The active principles from many traditional medicines have been extracted from plants, the curative agents identified and their mechanisms of action determined. Plant based medicines are typically well tolerated, with less severe side effects as well as a smaller range of side effects. In contrast, while synthetic drugs can be highly effective, their use is often hampered by severe side effects. Additionally, while synthetic pharmaceuticals are based upon single chemicals, many phytomedicines exert their beneficial effects through the additive or synergistic action of several chemical compounds acting at single or multiple target sites associated with a physiological process.

As pointed out by Tyler (1999), this synergistic or additive pharmacological effect can be beneficial by eliminating the problematic side effects associated with the predominance of a single xenobiotic compound in the body. In this respect, Kaufman et al. (1999) extensively documented how synergistic interactions underlie the effectiveness of a number of phytomedicines. A more recent study with additional demonstration concerning a phytomedicine's synergistic effect—Echinacea—is provided by Dalby-Brown et al, 2005. This theme of multiple chemicals acting in an additive or synergistic manner likely has its origin in the functional role of secondary products in promoting plant survival. For example, in the role of secondary products as defense chemicals, a mixture of chemicals having additive or synergistic effects at multiple target sites would not only ensure effectiveness against a wide range of herbivores or pathogens but would also decrease the chances of these organisms developing resistance or adaptive responses (Kaufman et al., 1999; Wink, 1999). Conclusion: On one hand, synthetics may have the required efficacy for disease treatments; however this can be marred by severe side effects. On the other hand, despite the excellent medicinal qualities of many plants, they are individually insufficient to take chronic degenerative diseases into remission. However, there is mounting evidence which demonstrate that medical plants contain synergistic and/or side-effect neutralizing combinations (Gilani and Rahman, 2005). Thus, what are needed in the art are better treatment regimes with improved patient tolerance, while providing sufficient efficacy.

### SUMMARY OF THE INVENTION

A number of known beneficial plants and nutraceuticals were classified according to their capacity to enhance the three main elements that support overall health, in chronic degenerative diseases: Energy (E), Bio-intelligence (I) and Organization (O). A synergistic effect is expected when all three categories of herbs and nutraceuticals (E, I, O) are included in a formulation.

Thus, on the case of hypertension, one embodiment of the invention provides an effective, natural composition for treating the aforementioned condition/disease. The composition can be used alone, or can be combined with simultaneous use of one or more pharmaceutical compositions.

### DETAILED DESCRIPTION OF THE INVENTION

‘Pharmaceutically acceptable excipients’ is used herein according to art accepted meanings, and includes those ingredients needed to formulate a medicine for mammalian use, including the use of gelatin capsules.

‘Synergistic’ or ‘synergy’ is used herein to mean that the effect is more than its additive property. In preferred embodiments, the synergy is at least 1.5, 2, 5, or 10 fold.

By use of ‘plants,’ what is meant herein is that the plant (or that portion with medicinal activity) is used whole, ground, or as an extract. Also included are purified active ingredients, and derivatives thereof. However, it is believed that the best efficacy of plants used herein is achieved with the use of the entire plant or its extracts, rather than with the use of isolated active ingredients.

Further, although plants are named here according to commonly used nomenclature, with improving taxonomy plants are often reclassified. Whenever a plant is referenced, it includes related species with similar active ingredients.

The following examples are illustrative only, and should not serve to unduly limit the invention.

### Energy Enhancers.—

Panax ginseng (Chinese ginseng, panax, ren shen, jintsam, ninjin, Asiatic ginseng, Japanese ginseng, Oriental ginseng, Korean red ginseng) The main active components are ginsenosides (protopanaxadiols and protopanaxatriols types) these have been shown to have a variety of beneficial effects, including immune modulator, anti-inflammatory, antioxidant, and anticancer effects. They also confer energizing properties because they increase ATP synthesis. Various—recent—studies validate Panax role as a hypotensive.

For example, Panax ginseng's extract (G115) effect on angiotensin-converting enzyme (ACE) and NO activity in cultured human endothelial cells from umbilical veins were investigated. Conclusion: Extract of Panax ginseng inhibits ACE activity but does not affect NO production (Persson I A, Dong L, Persson K. Effect of Panax ginseng extract (G115) on angiotensin-converting enzyme (ACE) activity and nitric oxide (NO) production. J Ethnoprarmacol.2006; 105:321-5). In another study evidence was provided on panax Re ginsenosides capability to activate endothelial NO synthase (eNOS) to release NO, resulting in an activation of the slowly activating delayed rectifier K(+) current, protecting against ischemia-reperfusion injury (Furukawa T et al. Ginsenoside Re, a main Phytosterol of Panax ginseng, Activates Cardiac Potassium Channels via a Non-Genomic Pathway of Sex Hormones. Mol. Pharmacol. Epub 2006, Sep. 19). Another study expands from Korean Red ginseng's (KRG) known capability to enhance the release of nitric oxide (NO) from endothelial cells of rats' aorta and reduce blood pressure in animals to suggesting that KRG may be useful for the treatment of hypertension and pulmonary vascular obstruction in humans (Han K et al. Korean Red Ginseng water extract increases nitric oxide concentrations in exhaled breadth. Nitric Oxide. 2005; 12:159-62). Panax ginseng provides at least 86 active principles in a single therapeutic.

Coenzyme Q10 (CoQ10), also known as ubiquinone or ubiquinol, is a biologically active quinone with an isoprenoid side chain, related in structure to vitamin K and vitamin E. CoQ is found in the membranes of endoplasmic reticulum, peroxisomes, lysosomes, vesicles and notably the inner membrane of the mitochondrion where it is an important part of the electron transport chain. CoQ is also essential in the formation of the apoptosome along with other adapter proteins. The loss of trophic factors activates pro-apoptotic enzymes, causing the breakdown of mitochondria. Because of its ability to transfer electrons and therefore act as an antioxidant, Coenzyme Q has become a valued dietary supplement. Young people are able to make Q10 from the lower numbered ubiquinones such as Q6 or Q8. The sick and elderly may not be able to make enough, thus Q10 becomes essential later in life or in illness. Supplementation of Coenzyme Q10 is a common component of the ‘mito cocktail’ used to treat mitochondrial disorders and other metabolic disorders. Recent studies show that Coenzyme Q10 antioxidant properties benefit the body. The data available from studies in animal models and from human intervention studies are consistent with a benefit of CoQ on vascular function and blood pressure (Hodgson J M, Watts G F. Can coenzyme Q10 improve vascular function and blood pressure? Potential for effective therapeutic reduction in vascular oxidative stress. Biofactors. 2003; 18:129-36). Clinical benefit from administration of CoQ10 to patients with essential hypertension could be based upon correcting a deficiency in bioenergetics, and point to possible combination treatments with a form of CoQ and anti-hypertensive drugs (Yamagami T, Shibata N, Folkers K. Bioenergetics in clinical medicine. Studies on coenzyme Q10 and essential hypertension. Res Commun Chem Pathol Pharmacol.1975; 11:273-88). A double-blind and double-crossover trial conducted by administering CoQ10 and a matching placebo orally to two groups of patients having class III or IV cardiomiopathy (New York Heart Association criteria). Group A received CoQ10 and then placebo; group B received placebo and then CoQ10. For group A, significant increases in cardiac function occurred during CoQ10 treatment and then decreased during crossover to placebo. For group B, there was no change in cardiac function during placebo treatment. These patients, steadily worsening and expected to die within 2 years under conventional therapy, generally showed an extraordinary clinical improvement, indicating that CoQ10 therapy might extend the lives of such patients. This improvement could be due to correction of a myocardial deficiency of CoQ10 and to enhanced synthesis of CoQ10-requiring enzymes (Langsjoen P H, Vadhanavikit S, Folkers K. Response of patients in classes III and IV of cardiomiopathy to therapy in a blind and crossover trial with coenzyme Q10. Proc Natl Acad Sci USA. 1985; 82:4240-4).

Rhodiola rosea (Golden Root, Roseroot) consists mainly of phenylpropanoids (rosavin, rosin, rosarin (specific to R. rosea), phenyl-ethanol derivatives (salidroside, rhodioloside, tyrosol), flavanoids (catechins, proanthocyanidins, rodiolin, rodionin, rodiosin, acetylrodalgin, tricin), monoterpenes (rosiridol, rosaridin), triterpenes (daucosterol, beta-sitosterol), and phenolic acids (chlorogenic and hydroxycinnamic, gallic acids). It also contains organic acids (gallic, caffeic, and chlorogenic acids) and p-Tyrosol.

There are many species of Rhodiola, but it appears that the rosavins are unique to R. Rosea, and it is the preferred species. Rhodiola increases energy levels because it activates ATP synthesis and re-synthesis in mitochondria, stimulating reparative processes after intense exercise. It has vasodilatation properties by mu-opiate receptors activation in the cardiovascular system. It is a hypolipidemic, diminishing cholesterol and triglyceride levels. Extracts of pure R. rosea have anti-ACE-I inhibitory activity. This high ACE-I inhibitory activity indicates potential for hypertension management (Apostolidis E, Kwon Y I, Shetty K. Potential of cranberry-based herbal synergies for diabetes and hypertension management. Asia Pac J Clin Nutr. 2006; 15:433-41). Extracts of R. rosea have high angiotensin I-converting enzyme inhibitory activity (Kwon Y I, Jang H D, Shetty K. Evaluation of Rhodiola crenulata and Rhodiola rosea for management of type II diabetes and hypertension. Asia Pac J Clin Nutr.2006; 15:425-32). Adaptogenic, cardiopulmonary protective and central nervous system activities of R. rosea have been attributed primarily to its ability to influence levels and activity of monoamines and opioid peptides. R. rosea provides at least 28 active principles.

Bio-Intelligence modulators.—

Astragalus membranaceus (Huang-Qi, Huangqi) This plant contains three main types of active principles: isoflavones, which act as anti-oxidants; astragalans which act as anti-inflammatory; and astragalosides which increase arterial and coronary flow, and improve heart function. 3-Nitropropionic acid (NPA), a compound obtained from Astragalus species, elicited a dose-dependent relaxation of pre-contracted rabbit aortic rings. On the other hand the chronic oral administration of NPA in renal hypertensive dogs provoked a decrease in blood pressure. The present results indicate that NPA has vasodilator and antihypertensive properties. The arterial relaxation elicited by NPA was inhibited with methylene blue suggesting that it is a consequence of guanylate cyclase stimulation. NPA cardiac effects may be related with inhibition of beta-adrenergic mediated responses (Castillo C, Valencia I, Reyes G. An analysis of the antihypertensive properties of 3-nitropropionic acid, a compound from plants in the genus Astragalus. Arch Inst Cardiol Mex.1993; 63:11-6). Results of another study indicate that Astragalus membranaceus can produce vascular relaxation. Mechanism may include the inhibition of intracellular calcium ions release by the 1,4,5-triphosphate inositol-receptor-dependent pathway in vascular smooth muscle cells (Zhang B Q, Hu S J, Shan Q X. Relaxant effect of Astragalus membranaceus on smooth muscle cells of rat thoracic aorta. Zhejiang Da Xue Xue Bao Yi Xue Ban.2005; 34:65-8, 72). Astragalus has therapeutic effects on sodium and water retention, the mechanisms of which might be the improvement of cardiac and renal functions, partly correcting abnormal mRNA expressions of AVP system and AQP2, and ameliorating blunted renal response to ANP (Ma J, Peng A, Lin S. Mechanisms of the therapeutic effect of Astragalus membranaceus on sodium and water retention in experimental heart failure. Chin Med J (Engl). 1998; 111:17-23).

Ganoderma lucidum (Reishi, also G. tsugae, G. valesiacum, G. oregonense, G. resinaceum, G. pfezfferi, G. oerstedli, and G. ahmadii) is an edible fungus containing bitter triterpenoids (ganoderic acid), β-D-glucan, coumarins, alkaloids and ergosterols. The main active principles of this mushroom are sterols and beta-proteoglucans. Ganoderma also contains Angiotensin converting enzyme-inhibitory triterpenes. Ganoderma total sterols significantly reduce malondialdehyde content and reactive oxygen species production and increases superoxide dismutase activity; furthermore, the translocation of nuclear factor-kappa B and the production of interleukin-1beta and tumor necrosis factor alpha induced by hypoxia/reoxygenation is blocked, suggesting that Ganoderma total sterols might be useful in treating hypoxia/reoxygenation-induced oxidative stress and inflammatory responses. Superoxide dismutase might play a critical role in the effect of Ganoderma against hypoxia/reoxygenation injury. In addition, Ganoderma component GS-1 significantly attenuated the formation of reactive oxygen species (Zhao H B, Wang S Z, He Q H. Ganoderma total sterol (GS) and GS1 protect rat cerebral cortical neurons from hypoxia/reoxygenation injury. Life Sci.2005; 76:1027-37). The amino-polysaccharide fraction from G. lucidum protects against oxidative damage induced by reactive oxygen species. Significantly inhibits lipid peroxidation and shows inactivation of hydroxyl radicals and superoxide anions (Lee J M, Kwon H, Jeong H. Inhibition of lipid peroxidation and oxidative DNA damage by Ganoderma lucidum. Phytother Res.2001; 15:245-9). It has vasodilator effect and is useful in the treatment of Arterial Hypertension. It is hypolipidemic and anti-atherotic. Ganoderma decreased systolic and diastolic blood pressure, accompanied by an inhibition of renal efferent sympathetic nerve activity. It was concluded that the mechanism of hypotensive action of Ganoderma lucidum was due to its central inhibition of sympathetic nerve activity (Lee S Y, Rhee H M. Cardiovascular effects of mycelium extract of Ganoderma lucidum: inhibition of sympathetic outflow as a mechanism of its hypotensive action. Chem Pharm Bull (Tokyo).1990; 38:1359-64). Ganoderma provides at least 32 active principles in a single therapeutic.

Grifola frondosa (Maitake, Dancing Mushroom; also G. sordulenta, Polyporus umbellatus and Meripilus giganteus) contains the primary polysaccharide, β-D-glucan in the 1.3 and 1.6 forms. It also contains alpha glucan, lipids, phospholipids, and ergosterol. A study found that Maitake lowers Systolic blood pressure significantly over the three to six weeks of study. (Talpur N, Echard B, Dadgar A. Effects of Maitake mushroom fractions on blood pressure of Zucker fatty rats. Res Commun Mol Pathol Pharmacol.2002; 112:68-82). Previous studies have demonstrated that niacin-bound chromium, Maitake mushroom and (−)-hydroxycitric acid (HCA) can ameliorate hypertension. This study showed that animals treated with these substances significantly lowered blood pressures as compared to control. Treatment of animals with a combination of these three novel supplements resulted in a lower Systolic Blood Pressure (Talpur N, Echard B W, Yasmin T. Effects of niacin-bound chromium, Maitake mushroom fraction SX and (−)-hydroxycitric acid on the metabolic syndrome in aged diabetic Zucker fatty rats. Mol Cell Biochem. 2003; 252:369-77). Maitake mushroom has been reported to favorably influence hypertension. This study in animals concluded that Maitake mushroom have antihypertensive potential. The Maitake fraction may decrease Systolic blood pressure via alteration in the renin-angiotensin system. (Talpur N A, Echard B W, Fan A Y. Antihypertensive and metabolic effects of whole Maitake mushroom powder and its fractions in two rat strains. Mol Cell Biochem. 2002; 237:129-36). The D-fraction, the MD-fraction, and other extracts from Maitake, often in combination with whole maitake powder, may provide some benefit in the treatment of hypertension (Mayell M. Maitake extracts and their therapeutic potential. Altern Med. Rev.2001; 6:48-60). The blood pressure of spontaneously hypertensive rats (SHR) were significantly reduced by Maitake feeding. The results suggest that dietary Maitake mushroom reduce the blood pressure (Kabir Y, Kimura S. Dietary mushrooms reduce blood pressure in spontaneously hypertensive rats (SHR). J Nutr Sci Vitaminol (Tokyo).1989; 35:91-4). Maitake decreased the blood pressure. The results suggest that dietary mushrooms prevent blood pressure increase in hypertension (Kabir Y, Yamaguchi M, Kimura S. Effect of shiitake (Lentinus edodes) and maitake (Grifola frondosa) mushrooms on blood pressure and plasma lipids of spontaneously hypertensive rats. J Nutr Sci Vitaminol (Tokyo).1987; 33:341-6). This phytomedicine provides at least 6 active ingredients.

Organizational improvers.—

Allium cepa L. (Liliaceae)—(Onion, Shallot, Basal, basl, cebolla, cebolla morada, cepa bulb, cepolla, cipolla, cu hanh, hom hua yai, hom khaao, hom yai, hu-t'sung, hu t'sung t'song, hua phak bhu, i-i-bsel, kesounni, khtim, Kilchenzwiebel, l'oignon, loyon, Madras oignon, oignon, palandu, piyaj, piyaz, pyaz, pyaaz, ralu lunu, red globe onion, sibuyas, Spanish onion, tamanegi, umbi bawang merah, vengayan, yellow Bermuda onion, white globe onion, Zwiebel). Sulfur- and non-sulfur-containing chemical constituents have been isolated from Bulbus Allii Cepae; the sulfur compounds are the most characteristic. The organic sulfur compounds of Bulbus Allii Cepae, including the thiosulfinates, thiosulfonates, cepaenes, S-oxides, S,S_-dioxides, monosulfides, disulfides, trisulfides, and zwiebelanes occur only as degradation products of the naturally occurring cysteine sulfoxides. When the onion bulb is crushed, minced, or otherwise processed, the cysteine sulfoxides are released from compartments and contact the enzyme alliinase in adjacent vacuoles. Hydrolysis and immediate condensation of the reactive intermediate (sulfenic acids) form the compounds as indicated below. The odorous thiosulphonates occur only in freshly chopped onions. The thiosulphinates and cepaenes appear to be the active constituents of Bulbus Allii Cepae. It also contains other active principles such as allicin, arginine, ascorbic-acid, calcium, magnesium, potassium, quercetin, rutin and tryptophan.

The principal use of Bulbus Allii Cepae today is to prevent age-dependent changes in the blood vessels, and loss of appetite (German Commission E Monograph, Allii cepae bulbus. Bundesanzeiger,1986, 50:13). Inhibition of platelet aggregation and inhibition of thromboxane synthesis by Bulbus Allii Cepae has been demonstrated both in vitro and in vivo and in humans. Both ethanol and methanol extracts of Bulbus Allii Cepae demonstrated diuretic activity (De A, Ribeiro R et al. Acute diuretic effects in conscious rats produced by some medicinal plants in the state of Sao Paulo, Brazil. Journalof ethnopharmacology, 1988, 24:19-29). Antihyperlipidemic and anti-cholesterolaemic activities have been demonstrated in clinical studies. It contains 230 active principles in a single therapeutic.

Allium sativum (garlic): The most important chemical constituents reported are the sulfur compounds. It has been estimated that cysteine sulfoxides (e.g. alliin) and the non-volatile γ-glutamylcysteine peptides make up more than 82% of the total sulfur content of garlic. The thiosulfinates (e.g. allicin), ajoenes, vinyldithiins and sulfides, however, are not naturally occurring compounds. Rather, they are degradation products from the naturally occurring cysteine sulfoxide, alliin. When the garlic bulb is crushed, minced, or otherwise processed, alliin is released from compartments and interacts with the enzyme alliinase in adjacent vacuoles. Hydrolysis and immediate condensation of the reactive intermediate (allylsulfenic acid) forms allicin. Allicin itself is an unstable product and will undergo additional reactions to form other derivatives. It contains other active principles which explain its hypotensive properties such as: adenosine, arginine, ascorbic-acid, calcium, magnesium, potassium, quercetin, tryptophan and tyrosinase. Twenty patients with essential hypertension and matched normotensive controls were enrolled in this study. Both groups were given garlic for 2 months. These findings point out the beneficial effects of garlic supplementation in reducing blood pressure, and thereby, offering cardioprotection in essential hypertensives (Dhawan V, Jain S. Garlic supplementation prevents oxidative DNA damage in essential hypertension. Mol Cell Biochem. 2005; 275:85-94). A pilot study in 101 adult subjects showed that individuals whose blood pressures are on the lower side are more likely to consume more garlic in their diets (Statistically significant for systolic blood pressure) (Qidwai W, Qureshi R, Hasan S N. Effect of dietary garlic (Allium Sativum) on the blood pressure in humans-a pilot study. J Pak Med. Assoc.2000; 50:204-7). In a systematic review, including meta-analysis, of seven trials that compared the effect of garlic with that of placebo, three studies showed a significant reduction in systolic blood pressure and four in diastolic blood pressure. For diastolic blood pressure the corresponding reduction in the garlic-treated subjects was slightly smaller. Results suggest that garlic may be of clinical use in subjects with mild hypertension. Garlic was evaluated in this open-label study in nine patients with rather severe hypertension. Results indicate that garlic can reduce blood pressure (McMahon F G, Vargas R. Can garlic lower blood pressure? A pilot study. Pharmacotherapy. 1993; 13:406-7). A clinical randomized placebo-controlled, double-blind trial in forty-seven patients with mild hypertension showed that patients who took garlic supine diastolic blood pressure fell significantly. In the placebo group, no significant changes occurred. (Auer W, Eiber A, Hertkorn E. Hypertension and hyperlipidaemia: garlic helps in mild cases. Br J Clin Pract Suppl.1990; 69:3-6). It contains 183 active principles.

Angelica sinensis (Dong quai or Angelica, also Angelica archangelia, Angelica pubescens and Angelica sylvestris, Can qui, Angelica china, dangdanggui, dang gui, dong quai, duong qui, handanggui, hashyshat almalak, kara toki, langdu danggui, min-gui, tang-kuei, tangkuei, tâ`n q´ui) Contains terpenes (terpenes, mainly β-phellandrene, with β-bisabolene, β-caryophyllene, β-phellandrene, α- and β-pinene, limonene, linalool, borneol, acetaldehyde, menthadienes and nitromenthadienes), macrocyclic lactones (including tridecanolide, 12-methyl tridecanolide, pentadecanolide), phthalates (such as hexamethylphthalate), coumarins (especially furocoumarin glycosides such as marmesin and apterin), angelicin and byakangelicin derivatives (osthol, umbelliferone, psoralen, bergapten, imperatoren, xanthotoxol, xanthotoxin, oxypeucedanin and more), as well as various sugars, plant acids, flavonoids, and sterols. It also, contains alkyl phthalides (Ligustilide); terpenes, phenylpropanoids (ferulic acid) and benzenoids. It contains also other active principles which explain its hypotensive properties, such as: ascorbic-acid, calcium, magnesium and potassium. Ligustilide is widely used in China to treat some pathological settings such as hypertension.

The results of this study show that ligustilide significantly inhibited vascular smooth muscle cells proliferation and cell cycle progression. These findings suggest the antiproliferative effect of ligustilide was associated with the decrement of reactive oxygen species. Thus, ligustilide contribute to be the effective agent in preventing cardiovascular diseases (Lu Q, Qiu T Q, Yang H. Ligustilide inhibits vascular smooth muscle cells proliferation. Eur J. Pharmacol. 2006; 542(1-3):136-40). Angelica sinensis has been widely used as traditional Chinese medicine to treat some pathological settings such as atherosclerosis and hypertension. Results of this study show that Angelica significantly inhibited proliferation and protein synthesis of vascular smooth muscle cell. On the other hand, Angelica significantly increased nitric oxide production of vascular smooth muscle cell. Data suggest that Angelica markedly inhibited vascular smooth muscle cell proliferation by arresting G(1) to S progression, which may be associated with nitric oxide production (Hou Y Z, Zhao G R, Yuan Y J. Inhibition of rat vascular smooth muscle cell proliferation by extract of Ligusticum chuanxiong and Angelica sinensis. J Ethnopharmacol. 2005; 100:140-4). Ferulic acid is a phenolic compound contained in Angelica sinensis and other plants. After oral administration of ferulic acid to hypertensive rats, systolic blood pressure significantly decreased. Intravenous injection of ferulic acid dose dependently reduced carotid arterial pressure in anesthetized hypertensive rats. Furthermore, the depressor effect of intravenous ferulic acid was significantly attenuated by pretreatment with nitric oxide (NO) synthase inhibitor NG-nitro-L-arginine methyl ester. Data suggest that the hypotensive effect of ferulic acid is associated with NO-mediated vasodilation. (Suzuki A, Kagawa D, Fujii A. Short- and long-term effects of ferulic acid on blood pressure in spontaneously hypertensive rats. Am J Hypertens.2002; 15:351-7). Angelica provides 70 active principles.

Capsicum annum (Bell Pepper, Cherry Pepper, Cone Pepper, Green Pepper, Paprika, Sweet Pepper). Its antihypertensive principles are: alpha-linolenic-acid, arginine, ascorbic-acid, calcium, magnesium, potassium, tetramethyl-pyrazine, and tryptophan. It also contains Capsaicinoids (capsanthin, capsanthin-5,6-epoxide, capsiamide, capsianoside-a-f, capsianoside-i-iv, capsianside-a, capsidiol, capsochrome, capsolutein, and capsorubin), flavonoids, essential oils, vitamin C, vitamin B1, vitamin B2, Fe and Cu. The active principles which explain its anti-inflammatory properties are: 1,8-cineole, alpha-linolenic-acid, ascorbic-acid, caffeic-acid, capsaicin and caryophyllene, which act as antioxidants, scavenging free radicals and reducing lipid peroxidation in cellular membranes (Howard L R, Talcott S T, Brenes C H, Changes in phytochemical and antioxidant activity of selected pepper cultivars—Capsicum species—as influenced by maturity. J Agric Food Chem. 2000; 48:1713-20). This phytomedicine provides at least 306 active principles.

Coleus forskohlii BRIQ (Lamiaceae) Active constituents: The labdane diterpene forskolin, derived from the root of the plant, is the primary constituent of clinical interest. Discovered by Western scientists in 1974 it was initially referred to as coleonol. Since that time, as other coleonols and diterpenoids have been identified, the name was changed to forskolin. Forskolin is responsible for virtually all pharmacological activities attributed to Coleus forskohlii; extracts of this constituent have been used in nearly all existing studies. There is evidence, however, that other plant constituents, such as volatile oils and other diterpenoids and coleonols, may contribute to the pharmacological activity and absorption of forskolin. Detailed analysis reveals approximately 20 constituents in various parts of the plant, but forskolin and other coleonols are present only in the root portion. Forskolin's primary mode of action is to increase cyclic adenosine monophosphate (cAMP) and cAMP-mediated functions, via activation of the enzyme adenylate cyclase. Forskolin has been shown to increase cAMP formation in all eukaryotic cells, without hormonal activation of adenylate cyclase. Forskolin's potentiation of cAMP in turn lowers blood pressure, inhibits platelet aggregation, promotes vasodilation, and stimulates lipolysis in fat cells. Animal and clinical studies demonstrate forskolin significantly lowers blood pressure via relaxation of vascular smooth muscle. In a small study of seven patients with dilated cardiomiopathy, forskolin significantly reduced diastolic blood pressure without increasing myocardial oxygen consumption; left ventricular function also improved. In a similar study, forskolin given to dilated cardiomiopathy patients resulted in decreased vascular resistance and a 19-percent improvement in left ventricle contractility.

Heart rate increased an average of 16 percent in study patients. Subjects also exhibited a 20-percent reduction in arterial pressure accompanied by symptomatic flush. Forskolin provides at least 20 active principles.

Crataegus oxyacantha (Hawthorn, also C. monogyna) contains mainly flavonoids (such as flavonoglycosyls, hyperoside, rutin, flavonol, kaempferol and quercetin) and oligomeric procyanidins (l-epicatechol), which relax arterial and decrease peripheral vascular resistance; amines (phenyletylamine, tyramine, O-methoxyphenethylamine); flavones (apigenin, luteolin) derivatives; vitexin glycosides, tannins, saponins, and cyanogenetic glycosides. Vasodilator: Crataegus extract induces an endothelium-dependent, NO-mediated vasorelaxation via eNOS phosphorylation (Brixius K, Willms S, Napp A. Crataegus Special Extract WS((R)) 1442 Induces an Endothelium-Dependent, NO-mediated Vasorelaxation via eNOS-Phosphorylation at Serine 1177. Cardiovasc Drugs Ther. Epub Jun. 21, 2006). Procyanidins of Crataegus caused endothelium-dependent relaxation which was associated with the production of cyclic GMP. Procyanidins of Crataegus may be responsible for the endothelium-dependent nitric oxide-mediated vascular relaxation, possibly via activation of tetraethylammonium-sensitive K+ channels (Kim S H, Kang K W, Kim K W. Procyanidins in crataegus extract evoke endothelium-dependent vasorelaxation in rat aorta. Life Sci. 2000; 67:121-31). Hawthorn flavonoids protect endothelial cell from hypoxia partly through its regulative effect on NO and calcium ion levels. Flavonoids and proanthocyanidins from Crataegus oxyacantha/C. monogyna demonstrated inhibitory activity of Angiotensin Converting Enzyme (ACE) (Lacaille-Dubois, Franck U, Wagner H. Search for potential angiotensin converting enzyme (ACE)-inhibitors from plants. Phytomedicine. 2001; 8:47-52). A randomized controlled trial showed the hypotensive effect of hawthorn in patients with diabetes taking medication. (Walker A F, Marakis G, Simpson E. Hypotensive effects of hawthorn for patients with diabetes taking prescription drugs: a randomised controlled trial. Br J Gen Pract. 2006; 56:437-43). Hawthorn (Crataegus laevigata) leaves, flowers and berries are used by herbal practitioners in the UK to treat hypertension in conjunction with prescribed drugs.

This randomized multicenter controlled clinical trial in 79 patients with type 2 diabetes showed a significant group difference in mean diastolic blood pressure reductions: the hawthorn group showed greater reductions than the placebo group. This is the first randomised controlled trial to demonstrate a hypotensive effect of hawthorn in patients with diabetes taking medication (Walker A F, Marakis G, Simpson E. Hypotensive effects of hawthorn for patients with diabetes taking prescription drugs: a randomised controlled trial. Br J Gen Pract. 2006; 56:437-43). Hawthorn (Crataegus) may play a role in the prevention and treatment of cardiovascular diseases such as hypertension, and in particular, congestive heart failure. Evidence is accumulating that hawthorn may induce hypotensive effects. These beneficial effects may in part be due to the presence of antioxidant flavonoid components (Chang W T, Dao J, Shao Z H. Hawthorn: potential roles in cardiovascular disease. Am J Chin Med. 2005; 33:1-10). Hawthorn is a fruit-bearing shrub with a long history of use in the treatment of cardiovascular and other disorders. Today, hawthorn is used primarily for various cardiovascular conditions. The cardiovascular effects are believed to be the result of ability to increase the integrity of the blood vessel wall and improve coronary blood flow, and positive effects on oxygen utilization. Flavonoids are postulated to account for these effects (Rigelsky J M, Sweet B V. Hawthorn: pharmacology and therapeutic uses. Am J Health Syst Pharm.2002; 59:417-22). In a clinical randomized controlled trial in 36 mildly hypertensive subjects, factorial contrast analysis showed a promising reduction in the resting diastolic blood pressure in the 19 subjects who were assigned to the hawthorn extract, compared with the other groups. Furthermore, a trend towards a reduction in anxiety was also observed in those taking hawthorn compared with the other groups (Walker A F, Marakis G, Morris A P. Promising hypotensive effect of hawthorn extract: a randomized double-blind pilot study of mild, essential hypertension Phytother Res. 2002; 16:48-54). Current uses of Crataegus oxycantha include treatment for hypertension (Miller A L. Botanical influences on cardiovascular disease. Altern Med. Rev.1998; 3:422-31). The results of this study suggest that hawthorn contains active components which cause arterial vasorelaxation.

Nitric oxide but not other endothelium-derived vasoactive factors were probably involved in the relaxation induced by hawthorn extract (Chen Z Y, Zhang Z S, Kwan K Y. Endothelium-dependent relaxation induced by hawthorn extract in rat mesenteric artery. Life Sci.1998; 63:1983-91). This placebo-controlled randomized double-blind study in 30 patients showed a mild reduction of the systolic and diastolic blood pressure in the Crataegus treated group (Leuchtgens H. Crataegus Special Extract WS 1442 in NYHA II heart failure. A placebo controlled randomized double-blind study] Fortschr Med. 1993; 111:352-4).

Eucommia ulmoides OLIV. (Eucommiaceae) (Du Zhong, Gutta-Percha Tree, Tu Chung, Propolis). The bark contains: Gutta-Percha (Balata); Lignans; Lignon glycosides; Syringaresinols; Olivils; Medioresinols; Coniferol alcohol; Ridoids; Eucommiol; Eucommioside; Genipin; Deoxyeucommiol; Geniposide; Geniposidic acid; Aucubin; Ajugoside; Reptoside; Asperuloside; Epieucommiol; Phenols; Catechol; Vanillic acid; Caffeic acid; Chlorogenic acid; Methyl chlorogenate; Eugenoside; Coniferin; Ulmoprenol; Kaempferol; Tartaric acid; Galactitol; Triacontanol Nonacosane; Geniposidic acid; b-sitosterol; Daucosterol; Straight Betulin; Betulinic acid; Ursolic acid; Lysine; Tryptophane; Methionine; Threonine; Valine; Leucine; Isoleucine; Glutamic acid; Cystisine; Histidine; Arginine; Chaintriterpenoid ethanol; 3-hydroxyphenylalanine; 3,4-dihydroxyphenylalanine. It also contains minerals which contribute to reduce arterial hypertension such as: calcium, magnesium and potassium. E. ulmoides extract was effective in reducing systolic blood pressure in hypertensive rats (Lang C, Liu Z, Taylor H W. Effect of Eucommia ulmoides on systolic blood pressure in the spontaneous hypertensive rat. Am J Chin Med. 2005; 33:215-30). A 4-week diet of propolis or Eucommia uloides resulted in significant reductions in systolic blood pressure in hypertensive rats. Results suggest that propolis produces an antihypertensive effect that may be mediated by potentiation of acetylcholine-induced vasodilatation (Kubota Y, Umegaki K, Kobayashi K. Anti-hypertensive effects of brazilian propolis in spontaneously hypertensive rats. Clin Exp Pharmacol Physiol. 2004; 31:S29-30). The vasorelaxant effects of E. ulmoides Oliv. on arteries was found to be entirely endothelium-dependent and nitric oxide (NO)-mediated.

Results offer a plausible mechanistic basis for the vasorelaxing action of E. ulmoides, which may account for its well-documented antihypertensive action. (Kwan C Y, Zhang W B, Deyama T. Endothelium-dependent vascular relaxation induced by Eucommia ulmoides Oliv. bark extract is mediated by NO and EDHF in small vessels. Naunyn Schmiedebergs Arch Pharmacol.2004; 369:206-11. This plant (and other closely related species) is commonly used in Chinese herbalism, where it is considered to be one of the 50 fundamental herbs. The aerial parts of the plant are alternative, antibacterial, antifungal, depurative, diuretic, emmenagogue, hypotensive, vasodilator and vulnerary (Handbook of Chinese Herbs and Formulas. by Yeung. Him-Che. Institute of Chinese Medicine, Los Angeles 1985). It contains at least 52 active principles.

Ginkgo biloba (Ginkgo) contains ginkgolides, bilobalides, bioflavonoids and flavone glycosides. Flavone glycosides include quercetin, 3-methylquercetin and kaempferol. Quercetin, myrcetin and the rest of the flavonoid fraction of the extract have antioxidant and free radical scavenger effects. The flavonoids increase blood flow. Their antioxidant properties and membrane stabilizing activity increase the tolerance to hypoxia. They improve cellular metabolism and protect against the damage caused by ischemia. Ginkgolide B is a powerful inhibitor of platelet activating factor (PAF), binding to its membrane receptors, and antagonizing platelet aggregation, which is one of the mechanisms that reduces arterial pressure. Similarly, it has anti-inflammatory effect by decreasing vascular permeability, and has vasodilator activity by inhibiting the liberation of thromboxane B2 and prostaglandins. Controlled double blind clinical studies conclusively demonstrate the effectiveness of Gingko biloba in treating peripheral arterial insufficiency. Other active principles which contain hypotensive such as: quercetin, tannin, amentoflavone. One clinical randomized double blind placebo controlled trial study in 70 healthy young volunteers showed that Ginkgo biloba reduced stress-induced rise in blood pressure without affecting the heart rate, providing evidence that Ginkgo has an inhibitory action on blood pressure (Jezova D, Duncko R, Lassanova M. Reduction of rise in blood pressure and cortisol release during stress by Ginkgo biloba extract (EGb 761) in healthy volunteers. J Physiol Pharmacol.2002; 53:337-48). It was demonstrated that Ginkgo biloba extract produced vasodilation via the nitric oxide synthesis and release by increasing the intracellular calcium level in vascular endothelial cells. Another study showed that the feeding of Ginkgo biloba significantly decreased systolic blood pressure. In the aortic preparations, the relaxation in response to acetylcholine was significantly potentiated by a Ginkgo biloba-containing diet. Results demonstrated that GBE reduced salt-related elevation of blood pressure and restored the impaired acetylcholine-induced vasodilation in aortic segments (Kubota Y, Tanaka N, Kagota S. Effects of Ginkgo biloba extract feeding on salt-induced hypertensive Dahl rats. Biol Pharm Bull.2006; 29:266-9). Administration of Ginkgo significantly decreased systolic blood pressure in hypertensive rats. In thoracic aortic preparations, diminished relaxation in response to acetylcholine was improved by a Ginkgo-containing diet. The results of this study suggested that Ginkgo enhanced endothelium-dependent vasodilation and elevation of the endothelial intracellular Ca(2+) level, resulting in hypotension. This accelerative effect of Ginkgo on Ca(2+) mobilization seemed to be associated with restoration of impaired dilatory function induced by acetylcholine in endothelial cells. (Kubota Y, Tanaka N, Kagota S. Effects of Ginkgo biloba extract on blood pressure and vascular endothelial response by acetylcholine in spontaneously hypertensive rats. J Pharm Pharmacol.2006; 58:243-9). The results of this study indicate that Ginkgo decreases blood pressure and mediates strong antithrombotic and anti-oxidant effects. These pharmacological activities may contribute to the beneficial properties of ginkgo observed in clinical practice (Sasaki Y, Noguchi T, Yamamoto E. Effects of Ginkgo biloba extract (EGb 761) on cerebral thrombosis and blood pressure in stroke-prone spontaneously hypertensive rats. Clin Exp Pharmacol Physiol.2002; 29:963-7). Development of hypertension was attenuated in rats fed a Ginkgo biloba diet. In addition, an increase in heart weight, an indicator of sustained high BP, was inhibited significantly by feeding of the Ginkgo biloba diet. Decreases in 5-hydroxytryptamine content in platelets, a marker of platelet activation in vivo associated with hypertension, were also prevented by feeding of the Ginkgo biloba diet. Feeding of the Ginkgo biloba diet tended to inhibit increases in plasma urea nitrogen due to hypertension.

Results indicate that Ginkgo biloba has anti-hypertensive action (Umegaki K, Shinozuka K, Watarai K. Ginkgo biloba extract attenuates the development of hypertension in deoxycorticosterone acetate-salt hypertensive rats. Clin Exp Pharmacol Physiol.2000; 27:277-82). Ginkgo provides 59 active principles.

Leonurus heterophyllus (L. japonicus, Leonurus Artemisia, Leonurus cardiaca, Leonurus sibiricus, Motherwort, Chinese Motherwort, Yi Mu Cao, I-mu-ts´ao, yakumoso, ikmoch´o, yik mo cho, chong wei, kun cao, Agripalma, Marihuanilla, Stachys Artemisia). Since immemorial time the Chinese people have used various parts of motherwort for improving blood flow both by official physicians and herbal practitioners throughout the country as well as by villagers in isolated areas. All Leonurus species are thought to have similar properties. Germany's Commission E has authorized Motherwort for the treatment of heart disorders caused by anxiety and stress, as well as part of an overall treatment plan for hyperthyroidism. Its active principles are: alkaloids (stachydrine, leocardin, leonuridine, leonurine, leonurinine A-B, Leonuridine), Fumaric acid, 4-Guanidino-1-butanol, 4-Guanidino-butyric acid, Arginine, Stachyose, resin, fat, Lauric acid, Linoleic acid, phytosterols (β-sitosterol, stigmasterol), α-Amyrin, cyclic nonapeptides (cycloleonuripeptide E and F), labdane diterpenes (prehispanolone, leoheteronins A-E, leojaponin, 13-epi-preleoheterin, iso-preleoheterin), 12 natural bis-spirolabdane-type diterpenoids, (leoheteronones A-E, 15-epileoheteronones B, D, E), leopersin B, 15-epileopersin B, leopersin C, 15-epileopersin C, flavonoids (Rutin, quercetin, quercetin-derivatives, genkwanin, isolavandulifolioside, lavandulifolioside). It also contains other active principles which explain its hypotensive properties: ascorbic-acid, quercetin, rutin, tannin. Cycloleonuripeptide E and cycloleonuripeptide F from the fruits of Leonurus heterophyllus showed moderate vasorelaxant effects on rat aorta (Morita H., lizuka T., Gonda A. Cycloleonuripeptides E and F, Cyclic Nonapeptides from Leonurus heterophyllus. J. Nat. Prod. 2006; 69: 839-841). Lavandulifolioside from Leonurus cardiaca decreases blood pressure (Milkowska-Leyck K, Filipek B, Strzelecka H. Pharmacological effects of lavandulifolioside from Leonurus cardiaca J Ethnopharmacol.2002; 80:85-90). A study demonstrated that Herba leonuri has antioxidant effects both in vitro and in vivo.

The antioxidant effects are exerted by selectively preserving the activities of superoxide dismutase and glutathione peroxidase, as well as depressing the formation of malondialdehyde, especially in the acute phase of acute Myocardial Infarct. Its effects of scavenging free radicals and inhibiting the formation of reactive oxygen species may play a key role in protecting the endogenous antioxidant system from oxidative stress in vivo (Sun J, Huang S H, Zhu Y C. Anti-oxidative stress effects of Herba leonuri on ischemic rat hearts. Life Sci. 2005; 76:3043-56). This phytomedicine contains at least 50 active principles.

Magnesium Its status has a direct effect upon the relaxation capability of vascular smooth muscle cells and the regulation of the cellular placement of other cations important to blood pressure—cellular sodium:potassium (Na:K) ratio and intracellular calcium (iCa(2+)). As a result, magnesium has both direct and indirect impacts on the regulation of blood pressure and therefore on the occurrence of hypertension. Hypertension occurs when cellular Na:K ratios become too high, a consequence of a high sodium, low potassium diet or, indirectly, through a magnesium deficient state which causes a pseudo potassium deficit. Like wise, magnesium deficiency alters calcium metabolism, creating high iCa(2+), low serum calcium and low urinary calcium states even when calcium intake is adequate. High iCa(2+) and high cellular Na:K ratio both occur when cellular magnesium becomes too low and the Mg-ATP driven sodium-potassium pump and calcium pump become functionally impaired. Several studies on the effect of calcium on blood pressure need these added considerations of magnesium status to fully understand the impact of the Mg:Ca ratio as the primary cause of hypertension and other aspects of Syndrome X. Magnesium supplementation above 15 mmol per day are required to normalize high blood pressure in unmedicated hypertensive patients while 15 mmol per day will lower high blood pressure in patients treated with anti-hypertensive medications. In most humans, healthy blood pressure depends upon a balance of both Na:K and Mg:Ca ratios at both cellular and whole body levels which, in turn, require adequate, long-term intakes of nutritional magnesium (Rosanoff A. Magnesium and hypertension. Clin Calcium.2005; 15:255-60). 133 patients with essential hypertension (EH) and 147 healthy subjects were enrolled in this study. Thirty-five patients randomly received magnesium potassium supplementation and 32 patients received lacidipin as a control. It was found that arterial compliance was significantly lower in EH patients compared with healthy subjects. On K+ and Mg2+ supplementation, systolic and diastolic BP decreased and C1 and C2 compliance values increased. Both large arterial compliance and small arterial compliance were decreased in essential hypertension patients. In essential hypertension patients, magnesium and potassium supplementation could improve small arterial compliance (Wu G, Tian H, Han K. Potassium magnesium supplementation for four weeks improves small distal artery compliance and reduces blood pressure in patients with essential hypertension. Clin Exp Hypertens.2006; 28:489-97). Inhibition of cardiovascular muscle cell Na+, K-ATPase activity due to increased level of endogenous sodium potassium pump inhibitor (SPI) is involved in the mechanism of volume expanded (VE) experimental and human essential hypertension (HT). Diets fortified with very high potassium (K) or very high magnesium (Mg) decrease blood pressure (BP). The data of this study show that K and Mg have additive effects in preventing an increase in SPI, thus probably preventing the Blood Pressure increase (Pamnani M B, Bryant H J, Clough D L. Increased dietary potassium and magnesium attenuate experimental volume dependent hypertension possibly through endogenous sodium-potassium pump inhibitor. Clin Exp Hypertens.2003; 25:103-15).

Olea europaea (olive): Key active constituents are Secoiridoids (oleuropein, hydroxytyrosol) and Flavonoids (hesperidin, rutin, apigenin, apigenin-4′-O-rhamnosylglucoside, apigenin-7-O-glucoside, quercetin, quercetin-3-O-rhamnoside, luteolin, luteolin-4′-O-glucoside, luteolin-7-O-glucoside, kaempferol, chrysoeriol, and chrysoeriol-7-O-glucoside). It contains active principles which explain its hypotensive properties, such as: calcium, oleuropein, oleuropeoside, potassium, quercetin, rutin and verbascoside. In a study by the Service de Cardiologie, Hopital Militaire, Tunis, treating patients suffering with essential hypertension, Olea europaea L. aqueous extract was given to 30 patients (12 undergoing treatment for the first time, 18 with antihypertensive treatment). A statistically significant decrease in blood pressure was noted for all patients (Cherif S, et al, A clinical trial of a titrated Olea extract in the treatment of essential arterial hypertension. J Pharm Belg 1996; 51:69-71). Another study's results suggested that in addition to oleuropeoside as a vasodilator, at least one other principle in olive leaf is either a vasodilator or potentiates the relaxant effect of oleuropeoside (Zarzuelo A, et al, ‘Vasodilator effect of olive leaf’, Planta Med, 1991; 57:417-9). Olea europaea leaves prevented the development of severe hypertension and atherosclerosis in the experimental animals (Somova L I, Shode F O, Ramnanan P. Antihypertensive, antiatherosclerotic and antioxidant activity of triterpenoids isolated from Olea europaea, subspecies africana leaves. J Ethnopharmacol.2003; 84:299-305). Olive leaf extract showed a prophylactic effect against the rise in blood pressure induced by L-NAME. In rats previously rendered hypertensive by L-NAME and then treated with the extract, normalization of the blood pressure was observed. The findings confirm previous reports on the hypotensive effects of olive leaf (Khayyal M T, el-Ghazaly M A, Abdallah D M. Blood pressure lowering effect of an olive leaf extract (Olea europaea) in L-NAME induced hypertension in rats. Arzneimittelforschung. 2002; 52:797-802). The olive leafs contain no less than 30 active principles.

Pueraria lobata (Kudzu): Kudzu contains a compound called puerarin (an isoflavone glycoside). Kudzu root also contains genistein and daidzein. Tambien contiene principios activos antihipertensivos, tales como: calcium, magnesium, potassium and quercetin. Chinese studies suggest that kudzu helps normalize blood pressure. When a tea containing about eight teaspoons of kudzu root was given daily to 52 people for two to eight weeks, 17 people experienced marked decline in their blood pressure. In one study, a tea containing about eight teaspoons of kudzu root was given daily to 52 people for two to eight weeks. In 17 people, blood pressure declined markedly. Thirty others showed some benefit. Puerarin has decreased blood pressure by 15 percent in laboratory animals. With 100 times the antioxidant activity of vitamin E, puerarin also helps prevent heart disease and cancer. Puerarin decreased blood pressure clinically. The results indicated that puerarin reduces both systolic and diastolic blood pressure. (Yang G, Zhang L, Fan L. Anti-angina effect of puerarin and its effect on plasma thromboxane A2 and prostacyclin. Zhong Xi Yi Jie He Za Zhi. 1990; 10:82-4, 68). Puerarin can elevate the level of NO concentration and decrease blood pressure. Wang C, Wang X Y, Zhou S. Therapeutic effect of puerarin on rats with pre-eclampsia. Zhonghua Fu Chan Ke Za Zhi. 2006; 41:118-20). Puerarin can suppress the proliferation and DNA synthesis of vascular smooth muscle cells. This inhibitory effects of puerarin are closely related with the suppression of c-fos and bcl-2 protein, and partly related with the suppression of the TR mRNA expression. (Xu Y Z, Gao Y, Li P Z. Puerarin suppresses the proliferation of vascular smooth muscle cells and c-fos and bcl-2 protein expression. Zhongguo Zhong Yao Za Zhi. 2006; 31:490-3). Puerarin can inhibit L-type calcium current of rat ventricular myocytes. Which implies that puerarin takes part in anti-myocardial ischemia and anti-arrhythmics partly due to the inhibition of L-type calcium channel. (Guo X G, Chen J Z, Zhang X. Effect of puerarin on L-type calcium channel in isolated rat ventricular myocytes. Zhongguo Zhong Yao Za Zhi. 2004; 29:248-51). Puerarin completely relaxed the contractions induced by phenylephrine in endothelium-intact and endothelium-denuded rat aorta. The relaxant effect of puerarin was significantly inhibited by pretreatment of endothelium-denuded aorta with potassium channel antagonists tetraethylammonium, 4-aminopyridine. Conclusion: Puerarin induces an endothelium-independent relaxation in rat aortic rings. The mechanisms may involve the reduction in Ca2+ influx through the calcium channels operated by alpha-adrenergic receptor and the activation of the potassium channels (Kv and BKca) (Dong K, Tao Q M, Xia Q. Endothelium-independent vasorelaxant effect of puerarin on rat thoracic aorta. Zhongguo Zhong Yao Za Zhi. 2004; 29:981-4). Puerarin has blocking effect on L-type calcium channel in a concentration dependent manner (Qian Y, Li Z, Huang L. Blocking effect of puerarin on calcium channel in isolated guinea pig ventricular myocytes. Chin Med J (Engl). 1999; 112:787-9). Increasing NO content and NOS activity in tissues may be one of the mechanism for pharmaceutical action of Puerarin (Wu P, Zeng F, Ma H X. Study on effect of Puerarin on nitric oxide system in rats' tissue and its mechanism. Zhongguo Zhong Xi Yi Jie He Za Zhi. 2001; 21:196-8). A clinical trial showed that Puerarin might play an important role in regulating the imbalance of endothelin, renin activity and angiotensin II. (Li S M, Liu B, Chen H F. Effect of puerarin on plasma endothelin, renin activity and angiotensin II in patients with acute myocardial infarction. Zhongguo Zhong Xi Yi Jie He Za Zhi. 1997; 17:339-41). Pueraria contains 26 active principles.

Tilia europea (Tilia spp., Basswood, Lime, Linden, lime tree). Parts used: flowers. Main constituents: hesperidin, saponins, tannin, quercetin. Also: 2-phenylethanol, 2-phenylethyl-benzoate, 2-phenylethylphenylacetate, afzelin, alanine, alpha-pinene, astragalin, caffeic-acid, chlorogenic-acid, cis-trans-farnesol, cis-trans-farnesyl-acetate, cysteine, cystine, docosane, eicosane, eugenol, geraniol, geranyl-acetate, heneicosane, hentriacontane, heptacosane, hexacosane, isoleucine, isoquercitrin, kaempferitrin, kaempferol-3(p-coumaroylglucoside), kaempferol-3,7-dirhamnoside, kaempferol-3-gluco-7-rhamnoside, leucine, limonene, linalol, linalyl-acetate, mucilage, nerol, nerolidol, nonadecane, octacosane, octadecane, p-coumaric-acid, pentacosane, phenylalanine, quercetin-3-glucoside-7-rhamnoside, quercetin-rhamnoside-xyloside, quercitrin, serine, terpineol, tetracosane, tiliroside, tocopherol, triacontane, tricosane, vanillin Action: vasodilator, anxiolytic. Tilia flowers also contain magnesia, which has sedative, antispasmodic and vasodilation action. Results of another study shows that Tilia americana var. mexicana possesses anxiolytic and sedative activity similar to the better-studied species of European Tilia reinforcing its use as anxiolytic and sedative in traditional medicine (Aguirre-Hernandez E, Martinez A L, Gonzalez-Trujano M E. Pharmacological evaluation of the anxiolytic and sedative effects of Tilia americana L. var. mexicana in mice. J Ethnopharmacol. Epub 2006, Jul. 21). Tilia flowers contain at least 57 active principles.

Zingiber officinale Roscoe (Zingiberaceae) Ginger. Active Components: Contains oleoresin with essential oil and pungent substances. The essential oil has a variable composition: the principal components are sesquitherpenes such as a-zingiberen, a-curcumene, β-bisabolen, β-bisabolone, (EE)-a-pharnesene and β-sesquiphelandren, and monotherpenes like camphor, β-phelendren, geranial, neral and linalol. The pungent substances are gingerols and sogaols. They are non volatile phenilalcanones or phenilalcanonoles with chains of a different length, being the most important ones the (6)-gingerol and the (6) sogaol. The rhizome of ginger contains also diarylheptanoids: diphenylheptenones, diphenylheptanonoles, diphenylheptanodioles and their acetates. Other components are: starch (approximately 50%), ditherpenes, 6-gingesulphonic acid and monoacyl digalactosyl glycerols.

It also contains other active principles which explain its hypotensive properties, such as: alpha-linolenic-acid, arginine, ascorbic-acid, calcium, gamma-aminobutyric-acid, magnesium, potassium, quercetin and tryptophan. Ginger, a well-known spice plant, has been used traditionally in a wide variety of ailments including hypertension. The extract of ginger induces a fall in the arterial blood pressure of anesthetized rats. In rabbit thoracic aorta preparation, the extract relaxed the phenylephrine-induced vascular contraction. Ca2+ channel-blocking (CCB) activity was confirmed when the ginger extract shifted the Ca2+ dose-response curves to the right similar to the effect of verapamil. It also inhibited the phenylephrine control peaks, indicating that it acts at both the membrane-bound and the intracellular Ca2+ channels. When tested in endothelium-intact rat aorta, it again relaxed the K-induced contraction. The vasodilator effect of ginger was endothelium-independent because it was not blocked by L-NAME or atropine and also was reproduced in the endothelium-denuded preparations at the same dose range. These data indicate that the blood pressure-lowering effect of ginger is mediated through blockade of voltage-dependent calcium channels (Ghayur M N, Gilani A H. Ginger lowers blood pressure through blockade of voltage-dependent calcium channels. J Cardiovasc Pharmacol. 2005; 45:74-80). General pharmacological studies were performed on (6)-gingerol and (6)-shogaol which are the pungent constituents of ginger (Zingiber officinale Roscoe). In the cardiovascular system, both (6)-shogaol and (6)-gingerol produced depressor response at lower doses on the blood pressure (Suekawa M, Ishige A, Yuasa K. Pharmacological studies on ginger. I. Pharmacological actions of pungent constituents, (6)-gingerol and (6)-shogaol. J Pharmacobiodyn.1984; 7:836-48). Ginger contains at least 312 active principles.

### EXAMPLE 2

### Composition—Hypertensive Disorders

A particularly preferred composition is shown in Table 1. Ratios reflect concentration of active ingredient over the natural state. Amounts provided are mg of extract. Obviously, the amount should be increased where the strength is reduced, and vice versa.

TABLE 1
Herbaria

| Active Agent | Ratio | Amount (mg) |
|---|---|---|
| Energy enhancers | | |
| Panax ginseng root extract | 5:1 | 15.00 |
| Coenzyme Q10 (CQ10) | 1:1 | 8.44 |
| Rhodiola rosea root extract | 10:1 | 22.00 |
| Bio-Intelligence modulators | | |
| Astragalus membranaceus root extract | 10:1 | 45.35 |
| Ganoderma lucidum mushroom extract | 10:1 | 36.60 |
| Grifola frondosa mushroom extract | 10:1 | 27.40 |
| Organization improvers | | |
| Allium cepa L. | 1:1 | 80.40 |
| Allium sativum | 3:1 | 30.44 |
| Angelica sinensis | 5:1 | 54.80 |
| Capsicum annum | 1:1 | 91.40 |
| Coleus forskohlii | 5:1 | 9.14 |
| Crataegus oxycantha | 5:1 | 73.12 |
| Eucommia ulmoides | 10:1 | 36.60 |
| Ginkgo bilova | 50:1 | 5.62 |
| Leonurus heterophyllus | 4:1 | 22.85 |
| Magnesium (MgO) | 60% | 91.40 |
| Olea europaea | 4:1 | 68.50 |
| Pueraria lobata | 5:1 | 54.84 |
| Tilia europea | 4:1 | 22.85 |
| Zingiber officinale | 5:1 | 18.28 |
| Total | | 815 |

### EXAMPLE 3

### A Clinical Study of Formulation's Anti-hypertensive Effectiveness and Tolerance

The effect of this composition was examined through a 3 month long prospective, descriptive, multicenter study in 50 patients with arterial hypertension. The administration of the composition significantly reduced arterial pressure values in 90% of the patients. Only 2% of the study group (4 patients) observed mild secondary effects, which did not warrant the suspension of the treatment. The formula was considered an alternative which with a combination of diet, exercise and other treatments may produce an unexpectedly superior therapeutic answer to this disorder.

### EXAMPLE 4

### Principles for Selecting Synergistic Combinations

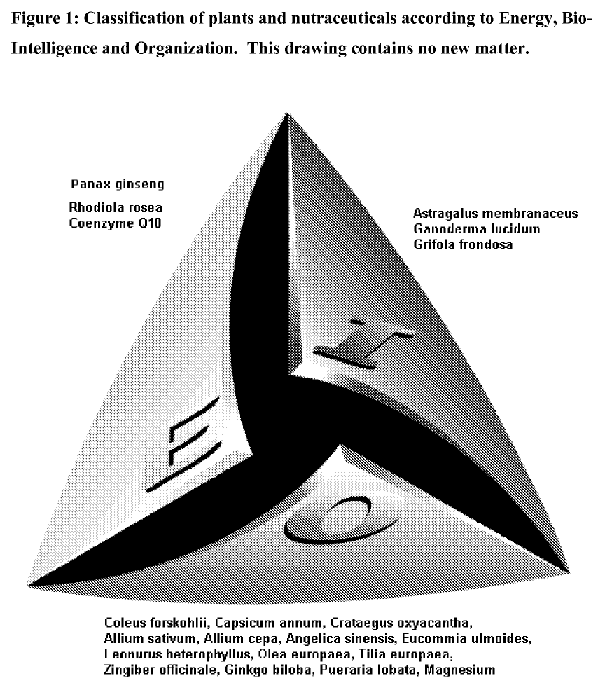

In order to explain the range of formulations encompassed by the invention, we have categorized beneficial plants and nutraceuticals into one of three groups, each of which should be present for synergistic effect. The classifications are: Energy, Bio-Intelligence and Organization. Plants and nutraceuticals classified under Energy are associated with ATP synthesis (such as the Krebs cycle, oxidative phosphorylation, beta-oxidation, etc.). Plants and nutraceuticals classified under Bio-Intelligence are those that regulate the neuroendocrine and immunological systems and cellular processes, thus controlling interactions between the various systems in the body. Finally, plants and nutraceuticals classified under Organization are those that relate to the structure and function of specific organs. Combinations of plants and nutraceuticals from these three classification groups have synergistic effect because they address each necessary component of cellular and organic health; providing the triangle—see FIG. 1—on which healing is fully supported.

FIG. 1, depicts the components-plants and/or nutraceuticals—which enhance Energy (E), modulate Bio-Intelligence (I) and improve Organization (O) sides of the aforementioned health triangle. That is, the components listed on the left hand view of FIG. 1, are the plants and/or nutraceuticals that enhance Energy. The plants and/or nutraceuticals in the right view are those that improve Organization. Finally, the plants and nutraceuticals at the bottom view of FIG. 1 modulate Bio-Intelligence.

An illustrative example of synergy in medicinal plants is an in vitro study that demonstrates how the activity of herbal Berberine alkaloids is strongly potentiated by the action of 5′-methoxyhydnocarpin (5′-MHC)—an active principle of another phytomedicine (denominated Hydnocarpus wightiana). It shows a strong increase of accumulation of berberine in the cells in the presence of 5′-MHC, indicating that this plant compound effectively disabled the bacterial resistance mechanism against the berberine antimicrobial, thus showing the synergy of both substances. Stermitz F R, et al., Synergy in a medicinal plant: antimicrobial action of berberine potentiated by 5′-methoxyhydnocarpin, a multidrug pump inhibitor. Proc Natl Acad Sci USA. 2000; 97:1433-7.

A further demonstration may be provided of synergistic effect on a molecular scale by studying the gene expression profile changes in response to various plant ingredients and combinations thereof. Experiments are already underway demonstrating the expression profile in response to the formulations. There is aid in this work because researchers have already begun studying the expression profiles of various medicinal plants, thus providing a database of knowledge from which to build. Eg., Gohil, et al., mRNA Expression Profile of a Human Cancer Cell Line in Response to Ginkgo Biloba Extract: Induction of Antioxidant Response and the Golgi System, Free Radic Res.2001; 33:831-849.

Finally there may be further presentation of gene expression results using whole-genome microarray analysis to demonstrate the formulation's capability to provide gene activation (upregulation or downregulation).

What is claimed is:

1) A phytoceutical composition, comprising plants or extracts or active ingredients derived from each of the following plants and nutraceuticals: Panax, Coenzyme Q10, Rhodiola, Astragalus, Ganoderma, Grifola, Allium, Angelica, Capsicum, Coleus, Crataegus, Eucommia, Ginkgo, Leonurus, Magnesium, Olea, Pueraria, Tilia and Zingiber together with pharmaceutically acceptable excipients.

2) The phytoceutical composition of claim 1, further comprising: Panax ginseng, Coenzyme Q10, Rhodiola rosea, Astragalus membranaceus, Ganoderma lucidum, Grifola frondosa, Allium cepa L., Allium sativum, Angelica sinensis, Capsicum annum, Coleus forskohlii, Crataegus oxycantha, Eucommia ulmoides, Ginkgo bilova, Leonurus heterophyllus, Magnesium, Olea europaea, Pueraria Lobata, Tilia europea and Zingiber officinale together with pharmaceutically acceptable excipients.

3) The phytoceutical composition of claim 2, comprising the relative amounts of ingredients shown in Table 1, and optionally including water or gelatin.

4) A method of treating disease comprising administering an effective amount of the composition of claim 3 to a patient sufficient to alleviate said disease.

5) The method of claim 4, wherein the diseases are hypertensive disorders and its symptoms.
